(19) United States
(12) Patent Application Publication
(10) Pub. No.: US 2003/0113351 A1
(43) Pub. Date: Jun. 19, 2003

(54) Lipid carrier

(76) Inventors: Andreas Fischer, Stockholm (SE); Christina Adde, Stockholm (SE); Bengt Herslöf, Stockholm (SE)

Correspondence Address: HOFFMANN & BARON, LLP, 6900 JERICHO TURNPIKE, SYOSSET, NY, 11791, US

(21) Appl. No.: 10/182,508

(22) PCT Filed: Mar. 5, 2001
(86) PCT No.: PCT/SE01/00461

(30) Foreign Application Priority Data
Mar. 6, 2000 (SE) 0000730-2

(51) Int. Cl. A61K 9/127; A61K 9/00; A61K 35/78

(52) U.S. Cl. 424/400; 424/731; 424/757; 424/776

(57) ABSTRACT

The invention refers to a lipid carrier composition for controlled release of a bioactive substance, which comprises at least one triglyceride oil, and at least one polar lipid selected from the group consisting of phosphatidylethanolamine and monohexosylceramide, and ethanol, which is characterised in that the carrier composition has the ability to form a cohesive structure, which structure is retained in an aqueous environment. The invention also refers to a pharmaceutical composition consisting of said lipid carrier and a bioactive substance dissolved or dispersed in the carrier, preferably an injectable composition.

### TECHNICAL FIELD

The present invention is related to a new lipid carrier composition for administration of biologically active materials, and in particular for sustained release of said bioactive materials in vivo.

### BACKGROUND OF THE INVENTION

For many types of drug substances there is a problem to create depot formulations in vivo, for example in the case of neuroleptic, antidepressive, anti-psychotic, antibiotic, antimicrobial, antidiabetic, and anti-Parkinson drugs. There are also many hormones and peptides, for example growth hormones and insulin, as well as cytostatic drugs, which suffer from the lack of suitable depot formulations.

There are today on the market several delivery systems for controlled and in particular sustained release of drug substances well-known to those skilled in the art. There are many examples of depot systems based on polymer systems from which the active compound is released through diffusion from a non-biodegradable matrix, or through biodegradation of the matrix, or, in the case of water soluble polymers, through dissolution of the polymer in the biological fluids. The non-biodegradable polymers do not undergo any significant change in the body. They are frequently used in implants, which often need to be eliminated by surgery. Also the biodegradable polymer systems are a potential risk of causing irritation to the site of implantation, which is also the case for water-soluble polymers during their dissolution and degradation in the body. The general disadvantages with polymeric systems, besides causing irritation, are also related to their capacity of incorporation, which in many cases is low and therefore restricted to highly potent drug substances. A practical problem is that a variety of polymers are needed in order to incorporate the many different drug substances and to meet their respective specific requirements in terms of incorporation level and release criteria.

Lipid oil systems, such as solutions or suspensions in triglyceride oils, so called fixed oils (USP XXIII), are also used for sustained release. Disadvantages with said systems are that only a limited number of compounds can be incorporated, including drugs which have been esterified with fatty acyl groups to pro-drugs, and that the release rate of such compounds cannot be influenced. This implies that these system are of limited value as parenteral depot systems. The use of other non-dispersed lipid carriers, i.e. oily vehicles, in pharmaceutical products is quite limited. The use of such systems for oral delivery is based on the self-emulsifying properties of the lipid system and an immediate release of the active compound in the gastrointestinal tract.

Other lipid systems than the oils and oily vehicles are dispersions, such as lipid emulsions and liposomes, which after intravenous administration offer only limited sustained release of incorporated drug substances. However, there are reports in the literature of intramuscularly or subcutaneously injected liposomes which do work as sustained release delivery systems, but the recognised difficulties are low encapsulation capacity and poor storage stability.

In order to avoid the disadvantages with dispersions a number of thermodynamically stable lipid systems have been developed. They are, however, based on the interaction of water with amphiphilic lipids to form stable liquid crystalline phases. Such systems have hitherto found very limited use in pharmaceutical applications.

### PRIOR ART

WO 84/02076, in the name of Fluidcarbon International, discloses control release compositions consisting of amphiphilic substances capable of forming a cubic liquid crystalline phase, such as monoglycerides, egg yolk phospholipids, and galactolipids, when in contact with water or aqueous systems.

WO 95/34287, in the name of GS Development AB, discloses a composition for slow release of biologically active materials based on a diacylglycerol, a phospholipid, and a polar liquid, which together form defined micellar or liquid crystalline systems.

WO 92/05771, in the name of Kabi Pharmacia AB, discloses a lipid particle forming matrix which can be used as a carrier for bioactive materials, from which lipid particles are formed spontaneously when interacting with aqueous systems. Said matrix consists of at least two lipid components, one is polar and amphiphilic and the other is nonpolar. One of the lipid components should also be bilayer forming. Phosphatidylcholine is used as the polar lipid in all examples. This system is self-dispersing in water, thus providing a more rapid release of the incorporated bioactive compound.

U.S. Pat. No. 4,610,868, in the name of The Liposome Company, Inc, refers to lipid matrix carriers, LMCs, which provide for sustained release of bioactive agents in vivo or in vitro. The LMCs are described as globular structures with a diameter ranging from about 500 to about 100,000 nm composed of a hydrophobic compound and an amphipathic compound. These globular structures are prepared in a cumbersome process involving dissolution of the lipid mixture in an organic solvent, agitation of the organic solution in an aqueous phase and evaporation of the organic solvent.

U.S. Pat. No. 5,912,271, in the name of Astra AB, refers to a new pharmaceutical preparation for topical administration comprising one or more local anaesthetic agents, a polar lipid, a triacylglycerol and optionally water. The polar lipid is preferably a sphingolipid or galactolipid, such as sphingolipids from milk or egg yolk, which are used in the examples.

WO 95/20945, in the name of Karlshamns Lipidteknik AB, relates to a lipophilic carrier preparation having a continuous lipid phase and comprising a polar lipid material, which is a galactolipid material consisting of at least 50% digalactosyldiacyglycerols, in combination with a non-polar lipid, and optionally a polar solvent.

There is still a need of a pharmaceutical carrier system, not comprising the disadvantages of the polymeric systems or the water containing lipid systems, respectively, but which enables a sustained release of a variety of drug substances with different chemical and physical properties in combination with a sufficient capacity for incorporation thereof.

### DESCRIPTION OF THE DRAWINGS

FIG. 1 shows the dissolution profiles obtained from carrier systems of the invention with bromothymol blue as a marker.

FIG. 2 shows the dissolution profiles obtained from carrier systems of the invention with safranine O as a marker.

### DESCRIPTION OF THE INVENTION

It has now surprisingly been found that a lipid carrier of the composition stated below has the ability to retain its cohesive structure with incorporated compounds in an aqueous environment, and therefore can be used for controlled release, such as sustained release, of an incorporated biologically active material. The lipids of the lipid carrier of the invention are based on lipid components, which are either normal components of the human cells and membranes, or are present in significant amounts in the human diet. This means that said lipids are biocompatible with human tissues and are metabolised in the same way as the corresponding endogenous lipids.

The invention refers to a lipid carrier composition for controlled release of a bioactive substance, comprising at least one triglyceride oil, and at least one polar lipid selected from the group consisting of phosphatidylethanolamine and monohexosylceramide, and ethanol, characterised in that the carrier composition has the ability to form a cohesive structure which is retained in an aqueous environment.

According to a preferred aspect of the invention the acyl groups of the polar lipid, which can be the same or different, are preferably derived from unsaturated or saturated fatty acids or hydroxy fatty acids having 12-28 carbon atoms.

The phosphatidylethanolamine can be obtained from all vegetable oil lecithin materials, for example soy lecithin, rape seed lecithin, sunflower lecithin, corn lecithin, cottonseed lecithin, but also from animal sources, for example egg yolk, milk (or other dairy materials), and animal organs or materials (brain, spleen, liver, kidney, erythrocytes), or any other source obvious to the person skilled in the art, but for practical reasons it is preferably obtained from soy lecithin and egg yolk. The chemical structure of a phosphatidylethanolamine, PE, can schematically be outlined as follows

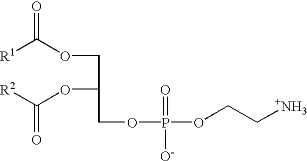

wherein R¹ and R² independently represent optionally substituted fatty acid residues.

According to a preferred aspect of the invention the phosphatidylethanolamine is egg-PE or dioleyl-PE.

The monohexosylceramide, CMH, also sometimes called monoglycosylceramide or cerebroside, can be of synthetic origin or obtained from milk (or other dairy products), animal organs or materials (brain, spleen, liver, kidney, erythrocytes), and plant sources. For practical reasons the monohexosylceramide is preferably obtained from milk or other dairy sources. In CMH from whey concentrate the majority of the fatty acyl chains linked to the amide nitrogen are of the compositions 22:0, 23:0 and 24:0. In CMH from plant sources the majority of the fatty acyl chains linked to the amide nitrogen are 2-hydroxy fatty acids. The chemical structure of a monohexosylceramide, CMH, can schematically be outlined as follows

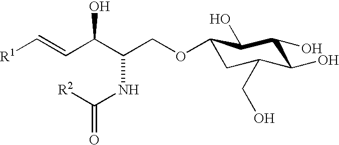

wherein R¹ and R² independently represent optionally substituted fatty acid residues.

The non-polar triglyceride oil, or in other words triacylglycerols, in the lipid carrier composition of the invention is preferably a triglyceride oil wherein the acyl groups are derived from unsaturated or saturated fatty acids or hydroxy fatty acids having 8-22 carbon atoms. The triglyceride oil can be selected from the group of natural vegetable oils consisting of, but not limited to, soybean oil, sesame oil, palm oil (or fractionated palm oils), safflower oil, evening primrose oil, sunflower oil, rape seed oil, linseed oil, corn oil, cottonseed oil, peanut oil, olive oil, castor oil (or fractionated castor oil, such as triricineolin) or from the group of semi-synthetic oils consisting of, but not limited to, medium chain triglyceride oil (also called fractionated coconut oil), acetylated monoglyceride oils, or from the group of animal oils, consisting of, but not limited to, butter oil, fish oil, or any mixture thereof, derived from any of these three groups. From a regulatory point of view the triglyceride oil is preferably selected from the group consisting of soybean oil, sesame oil, medium chain triglyceride oil, castor oil or a mixture thereof.

The sustained release properties of the lipid carrier system of the invention is depending on the lipid composition and can be controlled by selecting the proportions of the lipid components. Said proportions can also be selected to optimise the incorporation of specific bioactive materials, or to control the viscosity of the mixture. In order to obtain a lipid carrier composition, which is suitable for subcutaneous, intramuscular or intradermal injection, or for oral or ocular, dental or dermal administration, the following proportions of the lipid ingredients can be chosen: non-polar lipids 60-98%, polar lipids 0.1-40%, and ethanol 0.1-30%. In order to get an injectable preparation the triglyceride should preferably be liquid at ambient temperature.

The invention thus also refers to a lipid carrier consisting of 60-98% by weight of a triglyceride in combination with 0.1-40% by weight of at least one polar lipid selected from the group consisting of phosphatidylethanolamine and monohexosylceramide, and 0.1-30% by weight of ethanol.

Depending on the special features wanted of the lipid carrier, the content of polar lipid may be adjusted. The performance of the lipid carrier in aqueous environments is also depending on the choice of triglyceride, the content of ethanol and the presence of possible additives. In a lipid carrier composition having a high content of ethanol, the content of polar lipid may also have to be high for the carrier to stay cohesive in an aqueous solution.

The invention especially refers to a lipid carrier wherein the content of phosphatidylethanolamine, PE, is 5-40% by weight of the total carrier composition, preferably 10-25%.

According to another preferred aspect the invention refers to a lipid carrier wherein the content of monohexosylceramide, CMH, is 0.1-25% by weight of the total carrier composition, preferably 0.3-10%. The generally lower content of CMH compared to PE is due to the higher potency of CMH in giving the lipid carrier its cohesive structure in aqueous solutions.

One or more additives, such as glycerol, polyethylene glycols, propylene glycol, fatty alcohols, sterols, monoglycerides, tetraglycol, propylene carbonate and copolymers of polyethylene oxide and polypropylene oxide, or a mixture thereof, can be incorporated into the carrier in an amount of up to about 30% by weight of the total carrier composition. Said additives may have the ability to improve the solubility properties, and to alter the physical properties of the carrier. By changing the physical properties, such as polarity and viscosity, the release profile of the carrier may be modified. Any other additive, which can be incorporated into the carrier and does not negatively affect the active substance or the release thereof, can also be used.

The common feature of the different lipid compositions of the present invention is the coherent appearance of the carrier composition when brought into contact with different aqueous media. This has been observed in many different aqueous phases such as distilled water, 0.1 M HCl (pH 1), 0.1 M NaOH (pH 13), buffer solution that mimics the salt concentration and pH of human blood and interstitial fluids (20 mM Hepes, 150 mM NaCl, 0.01% w/w NaN₃, pH 7.4), solutions that mimic the salt concentration, pH and pepsin concentration of human gastric juice (2.0 g NaCl, 3.2 g pepsin, 80 ml 1M HCl, distilled water up to 1000 ml) and an acidic saline (70 mM NaCl, pH 1.0). The fact that the carrier composition of the present invention retains its cohesive, often gel-like appearance or structure, when poured or put into such diverse aqueous phases as described above makes it possible to use the carrier composition for controlled release in a number of different applications.

The invention refers to the use of a lipid carrier as described for the preparation of a depot formulation for injection for controlled release of a bioactive substance in vivo. Preferred ways of administration are by subcutaneous, intramuscular or intradermal injection.

The use of the invention for parenteral depot applications is obvious, but other uses are also obvious to the man skilled in the art. For example, the carrier can be used for oral delivery of drug substances. Because of the coherent appearance in aqueous solutions mimicing the human gastric juice it is furthermore convenient to think of applications where the carrier protects the drug substances in the gastric environment. Other possible uses for the lipid carrier of the invention are for taste masking of drugs in oral products. A specific aspect of the invention therefore is the use of a lipid carrier according to the invention for the preparation of an oral formulation for controlled release of a bioactive substance in vivo.

Slow release ocular and dental formulations, respectively, and other topical formulations, such as gels and ointments for dermal use, and formulations topically administered to the mucosa, as well as other applications where oils are used in pharmaceutical compositions, obvious to the man skilled in the art, are also possible uses. The invention also refers to the use of a lipid carrier as described for the preparation of an ocular, dental or dermal formulation for controlled release of a bioactive substance in vivo.

Depot formulations are of a general interest to the pharmaceutical industry. The invention also refers to a pharmaceutical composition for controlled release of a bioactive substance, which composition consists of a) a lipid carrier comprising at least one triglyceride oil in combination with at least one polar lipid selected from the group consisting of phosphatidylethanolamine and monohexosylceramide, and ethanol, which carrier has the ability to form a cohesive structure which is retained in an aqueous environment, and b) a bioactive substance dissolved or dispersed in said carrier.

A pharmaceutical composition according to the invention is especially characterised in that the lipid carrier consists of 60-98% by weight of a triglyceride in combination with 0.1-40% by weight of at least one of phosphatidylethanolamine and monohexosylceramide, and 0.1-30% by weight of ethanol, based on the total weight of the carrier, in addition to the bioactive substance.

A pharmaceutical composition of the invention can in addition contain one or more additives selected from the group consisting of glycerol, polyethylene glycols, propylene glycol, fatty alcohols, sterols, monoglycerides, tetraglycol, propylene carbonate and copolymers of polyethylene oxide and polypropylene oxide, and mixtures thereof.

The use of the carrier of the present invention is by no means limited to the ability of the carrier to dissolve the bioactive substance. Due to the semi-solid consistency, which can be obtained, of the carrier, it is possible to disperse and suspend solid crystalline and amorphous structures homogeneously into the carrier and prevent sedimentation upon storage.

The bioactive substance can be defined as a biologically active substance, which can be used within human or veterinary medicine, in cosmetics, food, and within agricultural applications.

The invention especially refers to a pharmaceutical composition wherein the bioactive substance is selected from the group consisting of neuroleptic, antidepressive, antipsychotic, antibiotic, antimicrobial, antitumour, and anti-Parkinson drugs, hormones, minerals and vitamins.

### EXAMPLES OF COMPOSITIONS

In the following examples the possibility to use different phosphatidylethanolamine and sphingolipid materials in the lipid carrier compositions is illustrated, as well as the necessity to include ethanol into the carrier to get a coherent structure. Pharmaceutical compositions are also illustrated.

The following materials were used in the examples:

Ethanol, 99.5%, from Kemetyl AB, Sweden;

Buffer solution of pH 7.4, consisting of 20 mM Hepes, 150 mM NaCl, 0.01% w/w NaN₃.

MCT oil (medium chain triglyceride oil) from Croda Oleochemicals, England, was used in the carrier composition examples.

### EXAMPLES OF CARRIER COMPOSITIONS WITH PHOSPHATIDYLETHANOLAMINE

The relative proportions, RP, of the carrier components MCT oil/PE/ethanol are given for each composition in % w/w. The following PE compounds were used in the examples:

Dipalmitoyl-PE from CHEMI S.p.A., Italy;

Distearoyl-PE from CHEMI S.p.A., Italy;

Dioleoyl-PE from CHEMI S.p.A., Italy;

Egg-PE was prepared from egg yolk by means of chromatographic fractionation to a purity of 95% (Scotia LipidTeknik AB, Sweden).

### Example 1

### Dipalmitoyl-PE (Comparative)

1.7372 g MCT oil was mixed with 0.1990 g DPPE and 0.0620 g ethanol in a sealed 10 ml glass vial. The mixture was stirred at 80° C. for 10 minutes without becoming homogeneous. When brought back to room temperature an inhomogeneous milky oil phase containing visible aggregates of DPPE was formed. RP: 86.9/10.0/3.1.

### Example 2

### Distearoyl-PE (Comparative)

1.6357 g MCT oil was mixed with 0.2944 g DSPE and 0.0418 g ethanol in a sealed 10 ml glass vial. The mixture was stirred at 80° C. for 10 minutes without becoming homogeneous. When brought back to room temperature an inhomogeneous milky oil phase containing visible aggregates of DSPE was formed. RP: 83.0/14.9/2.1.

### Example 3

### Dioleoyl-PE

1.6180 g MCT oil was mixed with 0.1862 g DOPE and 0.0545 g ethanol in a sealed 10 ml glass vial. The mixture was stirred at 80° C. for 10 minutes to form a homogeneous oil phase. When brought back to room temperature a macroscopically homogeneous, turbid oil phase of semi-solid consistency was formed ultimately. When put into the buffer solution the oil phase stayed coherent. RP: 87.1/10.0/2.9.

### Example 4

### Egg-PE

2.5633 g MCT oil was mixed with 0.4632 g egg-PE and 0.0656 g ethanol in a sealed 10 ml glass vial. The mixture was stirred at 80° C. for 5 minutes to form a homogeneous clear oil phase. When brought back to room temperature a macroscopically homogeneous, turbid oil phase of semi-solid consistency was ultimately formed. When put into the buffer solution the oil phase stayed coherent. RP: 82.9/15.0/2.1.

### Example 5

### Egg-PE Without Ethanol (Comparative)

2.6177 g MCT oil was mixed with 0.4620 g egg-PE in a sealed 10 ml glass vial. The mixture was stirred at 80° C. for 5 minutes to form a homogeneous oil phase. When brought back to room temperature a two phase system was formed. One phase of semi-solid consistency, and one phase of liquid oil. RP: 85.0/15.0/0.

The macroscopically, that is to the naked eye, homogeneous appearance of the carrier and the coherent behaviour when put into aqueous solutions has surprisingly not been found for all phosphatidylethanolamine (PE) materials tested. It has so far only been observed in mixtures comprising egg-PE and synthetic dioleoyl-PE.

### EXAMPLES OF CARRIER COMPOSITIONS WITH SPHINGOLIPID MATERIALS

In the following examples, the so far unique feature of monohexosylceramide, CMH, compared to other sphingolipid materials, when comprised into the carrier, is illustrated.

The relative proportions, RP, of the carrier components MCT oil/sphingolipids/ethanol are given for each composition in % w/w. The following sphingolipid compounds were used in the examples:

CMH (monohexosylceramide), prepared from whey concentrate by means of chromatographic fractionation to a purity of >98% (Scotia LipidTeknik AB);

CDH (dihexosylceramide), prepared from whey concentrate by means of chromatographic fractionation to a purity of >98% (Scotia LipidTeknik AB);

m-SL, milk sphingolipids containing approximately 70% sphingomyelin, 10% CMH and 10% CDH, prepared from whey concentrate by means of chromatographic fractionation (Scotia LipidTeknik AB);

Sphingomyelin, prepared from whey concentrate by means of chromatographic fractionation to a purity of >99% (Scotia LipidTeknik AB).

### Example 6

### CMH

1.8496 g MCT oil was mixed with 0.0600 g CMH and 0.1045 g ethanol in a sealed 10 ml glass vial. The mixture was stirred at 80° C. for 10 minutes to form a homogeneous oil phase. When brought back to room temperature a macroscopically homogeneous, turbid oil phase of semi-solid consistency was formed. When put into the buffer solution the oil phase stayed coherent. RP: 91.8/3.0/5.2.

### Example 7

### CMH Without Ethanol (Comparative)

1.9579 g MCT oil was mixed with 0.0604 g CMH in a sealed 10 ml glass vial. The mixture was stirred at 80° C. for 10 minutes to form a homogeneous oil phase. When brought back to room temperature a two phase system was formed. One phase of semi-solid consistency, and one phase of liquid oil. RP: 97.0/3.0/0.

### Example 8

### CDH (Comparative)

1.8025 g MCT oil was mixed with 0.0589 g CDH and 0.0985 g ethanol in a sealed 10 ml glass vial. The mixture was stirred at 80° C. for 10 minutes to form a homogeneous oil phase. When brought back to room temperature a two phase system was formed. One phase of semi-solid consistency, and one phase of liquid oil. RP: 92.0/3.0/5.0.

### Example 9

### m-SL (Comparative)

2.0280 g MCT oil was mixed with 0.0662 g milk sphingolipids and 0.1185 g ethanol in a sealed 10 ml glass vial. The mixture was stirred at 80° C. for 10 minutes to form a homogeneous clear oil phase. When brought back to room temperature an inhomogeneous oil phase of milk sphingolipid sediment in MCT oil was formed. RP: 91.7/3.0/5.4.

### Example 10

### Sphingomyelin (Comparative)

2.0606 g MCT oil was mixed with 0.0671 g sphingomyelin and 0.1098 g ethanol in a sealed 10 ml glass vial. The mixture was stirred at 80° C. for 10 minutes to form a homogeneous clear oil phase. When brought back to room temperature an inhomogeneous milky oil phase of sphingomyelin sediment in MCT oil was formed. RP: 92.1/3.0/4.9.

### EXAMPLES OF CARRIER COMPOSITIONS WITH MONOHEXOSYLCERAMIDE AND DIFFERENT ADDITIVES

In the following examples the ability to incorporate an additive into the carrier of the present invention is illustrated. Different additives were added to mixtures of different triglyceride oils, CMH, and ethanol in a sealed 10 ml glass vial. The CMH was the same as in Example 6. The relative proportions, RP, of the carrier components triglyceride oil/CMH/ethanol/additive are given for each composition in % by weight. The following oils and additives were used in the examples below:

Castor oil from Apoteksbolaget, Sweden;

Castor oil, extracted, (triricineolin), RRR, was prepared by Scotia LipidTeknik AB from castor oil from Karlshamns AB, Sweden;

Sesame oil from Croda Oleochemicals, England;

Glycerol, 99.8%, from Apoteksbolaget, Sweden;

Polyethylene glycol 400, for synthesis, from Kebo Lab AB, Sweden;

Polyethylene glycol 1000, for synthesis, from Kebo Lab AB, Sweden;

Polyethylene glycol 3000, for synthesis, from Kebo Lab AB, Sweden;

Propylene glycol, >99,5%, from Kebo Lab AB, Sweden;

Stearyl alcohol, >96%, from Kebo Lab AB, Sweden;

Cholesterol from Genzyme, England;

Monoglyceride, fractionated Akoline MCM, was prepared by Scotia LipidTeknik AB from Akoline MCM from Karlshamns AB, Sweden;

Tetraglycol from Sigma-Aldrich Sweden AB;

Propylene carbonate, 99%, from Sigma-Aldrich Sweden AB;

Lutrol F68 (Poloxamer 188) from BASF, Germany.

### Example 11

### Glycerol

1.8907 g MCT oil was mixed with 0.0735 g CMH, 0.1274 g ethanol and 0.3931 g glycerol. RP: 76.1/3.0/5.1/15.8.

### Example 12

### Glycerol

1.7984 g triricineolin was mixed with 0.0697 g CMH, 0.1254 g ethanol and 0.4413 g glycerol. RP: 73.9/2.9/5.2/18.1.

### Example 13

### PEG 400

2.3015 g triricineolin was mixed with 0.0893 g CMH, 0.2979 g ethanol and 0.2981 g polyethylene glycol 400. RP: 77.1/3.0/10.0/10.0.

### Example 14

### PEG 1000

1.5480 g triricineolin was mixed with 0.0599 g CMH, 0.1992 g ethanol and 0.1975 g polyethylene glycol 1000. RP: 77.2/3.0/9.9/9.9.

### Example 15

### PEG 3000

1.4735 g triricineolin was mixed with 0.0534 g CMH, 0.0955 g ethanol and 0.1834 g polyethylene glycol 3000. RP: 81.6/3.0/5.3/10.2.

### Example 16

### Propylene Glycol

1.5014 g triricineolin was mixed with 0.0542 g CMH, 0.0906 g ethanol and 0.1756 g propylene. RP: 82.4/3.0/5.0/9.6.

### Example 17

### Stearyl Alcohol

1.6449 g triricineolin was mixed with 0.0593 g CMH, 0.1068 g ethanol and 0.1965 g stearyl alcohol. RP: 81.9/3.0/5.3/9.8.

### Example 18

### Stearyl Alcohol

1.6752 g sesame oil was mixed with 0.0613 g CMH, 0.0995 g ethanol and 0.2038 g stearyl alcohol. RP: 82.1/3.0/4.9/10.0.

### Example 19

### Cholesterol

2.6898 g MCT oil was mixed with 0.1194 g CMH, 0.1467 g ethanol and 0.0309 g cholesterol. RP: 90.1/4.0/4.9/1.0.

### Example 20

### Cholesterol

2.4572 g MCT oil was mixed with 0.2315 g CMH, 0.1480 g ethanol and 0.0587 g cholesterol. RP: 84.9/8.0/5.1/2.0.

### Example 21

### Monoglyceride

1.7013 g triricineolin was mixed with 0.0615 g CMH, 0.2067 g ethanol and 0.1076 g monoglyceride. RP: 81.9/3.0/10.0/5.2.

### Example 22

### Tetraglycol

1.5517 g triricineolin was mixed with 0.0600 g CMH, 0.1948 g ethanol and 0.1988 g tetraglycol. RP: 77.4/3.0/9.7/9.9.

### Example 23

### Propylene Carbonate

1.5410 g triricineolin was mixed with 0.0591 g CMH, 0.2003 g ethanol and 0.2067 g propylene carbonate. RP: 76.8/2.9/10.0/10.3.

### Example 24

### Lutrol F68

1.6665 g castor oil was mixed with 0.0552 g CMH, 0.0920 g ethanol and 0.1246 g Lutrol F68. RP: 86.0/2.8/4.7/6.4.

The mixtures were stirred at 75-85° C. for 10 minutes to form a homogeneous oil phase. When the mixtures had been brought back to room temperature a macroscopically homogeneous, turbid oil phase of semi-solid consistency was formed in each case. When put into a buffer solution all oil phases stayed coherent. The macro-scopically homogeneous appearance of the carrier, comprising CMH, triglyceride oil, ethanol and optionally an additive, and the coherent behaviour of the same when put into aqueous solutions, has not been found for other sphingolipid materials tested.

### EXAMPLES OF PHARMACEUTICAL COMPOSITIONS

In the examples of pharmaceutical compositions below the following materials were used in addition to those previously mentioned:

Soybean oil from Karlshamns AB, Sweden;

MCT-oil (medium chain triglyceride oil) from Karlshamns AB, Sweden;

Castor oil from Karlshamns AB, Sweden;

Betamethasone dipropionate, USP XXIII; Supplier: Jucker Pharma, Sweden;

Cyclosporin A, USP XXIII; Supplier: Medial AG, Switzerland;

Medroxyprogesterone acetate, Batch ACL 973131 PL5; Apoteket Draken, Stockholm, Sweden;

Bacteriochlorin, SQN 400, Batch no CAR/99/00086; Scotia Pharmaceuticals, Stirling, Scotland;

Insulin, bovine, from Sigma-Aldrich Sweden AB;

Vitamin B12, 99%, from Sigma-Aldrich Sweden AB.

### Example 25

### Betamethasone

CMH/soybean oil/ethanol/betamethasone dipropionate, relative proportions 3.0/81.7/10.1/5.2% w/w.

1.7164 g soybean oil was mixed with 0.0625 g CMH, 0.1088 g betamethasone dipropionate and 0.2133 g ethanol in a sealed 10 ml glass vial. The mixture was stirred at 80° C. for 15 minutes to form a homogenous clear oil phase. The betamethasone dipropionate did not precipitate when the formulation was brought back to room temperature.

### Example 26

### Cyclosporin

CMH/soybean oil/ethanol/cyclosporin, relative proportions 3.0/81.6/10.3/5.2% w/w.

1.6014 g soybean oil was mixed with 0.0582 g CMH, 0.1012 g cyclosporin and 0.2013 g ethanol in a sealed 10 ml glass vial. The mixture was stirred at 80° C. for 15 minutes to form a homogenous clear oil phase. The cyclosporin did not precipitate when the formulation was brought back to room temperature.

### Example 27

### Medroxyprogesteron

CMH/MCT oil/ethanol/medroxyprogesteron acetate, relative proportions 3.0/82.4/10.4/4.2% w/w.

1.7644 g MCT oil was mixed with 0.0645 g CMH, 0.0900 g medroxyprogesteron acetate and 0.2227 g ethanol in a sealed 10 ml glass vial. The mixture was stirred at 80° C. for 15 minutes to form a homogenous clear oil phase. The medroxyprogesteron acetate did not precipitate when the formulation was brought back to room temperature.

### Example 28

### SQN 400

MCT oil/SQN 400/egg-PE/ethanol, relative proportions 51.1/6.0/28.7/14.2% w/w.

0.1058g SQN 400 was mixed with 0.900 g MCT oil at 70° C. for 15 min. 0.5045 g egg-PE was mixed with 0.250 g ethanol at RT. The two mixtures were mixed together in a sealed 10 ml glass vial. This mixture was stirred at 80° C. for 15 min to form a homogenous clear oil phase. The SQN 400 did not precipitate when the formulation was brought back to room temperature.

### Example 29

### Crystalline Insulin

Triricineolin/CMH/ethanol/insulin, relative proportions 82.8/3.1/9.3/4.8% w/w.

0.8520 g triricineolin was mixed with 0.0318 g CMH, 0.0962 g ethanol and 0.0493 g bovine insulin in a sealed 10 ml glass vial. The mixture was stirred at 800C for 10 minutes to form a homogeneous oil phase. When brought back to room temperature a macroscopically homogeneous, turbid oil phase of semi-solid consistency was formed. Examination of the sample with an optical microscope (Olympus CHS) revealed crystals of insulin evenly distributed throughout the carrier.

The mixture was left in the glass vial at room temperature. More than 17 weeks later the mixture was examined and the homogeneous, turbid, gel-like appearance of the oil phase was still observed, with no signs of sedimentation or partition of the constituents. Examination with the optical microscope showed the same even distribution as observed before.

### Example 30

### Compatibility with Hard Gelatin Capsules

In this example the compatibility of a pharmaceutical composition with hard gelatin capsules is illustrated. The following materials were used in addition to those previously mentioned:

MCT oil (medium chain triglyceride oil) from Croda Oleochemicals, England;

Hard gelatine capsules, Coni-Snap size 0, transparent, from Capsugel, Belgium.

1.8495 g triricineolin was mixed with 0.1022 g CMH and 0.1079 g ethanol containing 0.1% w/w vitamin B12 in a sealed 10 ml glass vial. The mixture was stirred at 80° C. for 10 minutes to form a homogeneous pink coloured oil phase. When brought back to room temperature a macroscopically homogeneous, pink coloured, turbid oil phase of semi-solid consistency was formed. The mixture was then filled in hard gelatin capsules, which were closed and placed in a sealed glass vial at 54% RH. The capsules were left at room temperature. More than 15 weeks later, the capsules were examined and showed no compatibility problems.

### SUSTAINED RELEASE EXAMPLES

First Experiments

In the following examples sustained release properties of lipid systems of the present invention are illustrated by the incorporation and release of methylene blue and bromothymol blue, respectively, as marker substances. The non-polar lipid was either soybean oil (from Karlshamns AB, Sweden), MCT-oil (medium chain triglyceride oil, from Karlshamns AB, Sweden), or castor oil (from Karlshamns AB, Sweden), the polar lipid was either CMH (monohexosylceramide from whey concentrate, Scotia LipidTeknik AB, Sweden) or PE (phosphatidylethanolamine from egg yolk, Scotia LipidTeknik AB, Sweden).

The following marker substances were used:

Methylene blue, grade “for microscopical staining”, from KEBO Lab AB, Sweden.

Bromothymol blue, grade “indicator”, from KEBO Lab AB, Sweden.

### Example 1(A)

1.9708 g soybean oil was mixed with 0.0644 g CMH and 0.1029 g ethanol containing 0.1% w/v methylene blue in a sealed 10 ml glass vial. The mixture was stirred at 80° C. for 10 minutes to form a homogeneous blue coloured oil phase.

### Example 2(B)

1.5441 g soybean oil was mixed with 0.4118 g PE and 0.1004 g ethanol containing 0.1% w/v methylene blue in a sealed 10 ml glass vial. The mixture was stirred at 80° C. for 5 minutes to form a homogeneous blue coloured oil phase.

### Example 3(C)

2.1246 g soybean oil was mixed with 0.1124 g ethanol containing 0.1% w/v methylene blue in a sealed 10 ml glass vial. The mixture was stirred at 80° C. for 5 minutes to form a homogeneous blue coloured oil phase.

### Example 4(D)

2.1846 g MCT oil was mixed with 0.1138 g ethanol containing 0.1% w/v methylene blue in a sealed 10 ml glass vial. The mixture was stirred at room temperature for 10 minutes to form a homogeneous blue coloured oil phase.

### Example 5(F)

1.8601 g fractionated castor oil was mixed with 0.0600 g CMH and 0.0966 ethanol containing 0.1% w/v methylene blue in a sealed 10 ml glass vial. The mixture was stirred at 80° C. for 20 minutes to form a homogeneous grey coloured oil phase.

### Example 6(F)

1.8668 g MCT oil was mixed with 0.0607 g CMH and 0.1075 ethanol containing 0.1% w/v methylene blue in a sealed 10 ml glass vial. The mixture was stirred at 80° C. for 10 minutes to form a homogeneous blue coloured oil phase.

### Example 7(G)

2.8418 g soybean oil was mixed with 0.0090 g CMH and 0.1445 ethanol containing 0.1% w/v methylene blue in a sealed 10 ml glass vial. The mixture was stirred at 80° C. for 10 minutes to form a homogeneous blue coloured oil phase.

### Example 8(H; Reference Solution)

0.024 g ethanol containing 0.1% w/v methylene blue was dissolved in 15 ml buffer solution and used as a reference solution, against which the release of methylene blue from mixtures A to G was compared.

### Example 9(I)

2.0302 g soybean oil was mixed with 0.0661 g CMH and 0.1214 g ethanol containing 0.1% w/v bromothymol blue in a sealed 10 ml glass vial. The mixture was stirred at 80° C. for 10 minutes to form a homogeneous yellow coloured oil phase.

### Example 10(J)

1.4468 g soybean oil was mixed with 0.3835 g PE and 0.0944 g ethanol containing 0.1% w/v bromothymol blue in a sealed 10 ml glass vial. The mixture was stirred at 80° C. for 5 minutes to form a homogeneous yellow coloured oil phase.

### Example 11(K)

2.1227 g soybean oil was mixed with 0.1115 g ethanol containing 0.1% w/v bromothymol blue in a sealed 10 ml glass vial. The mixture was stirred at 80° C. for 5 minutes to form a homogeneous yellow coloured oil phase.

### Example 12(L)

2.1242 g MCT oil was mixed with 0.1107 g ethanol containing 0.1% w/v bromothymol blue in a sealed 10 ml glass vial. The mixture was stirred at 80° C. for 5 minutes to form a homogeneous yellow coloured oil phase.

### Example 13(M)

1.7859 g fractionated castor oil was mixed with 0.0583 g CMH and 0.0990 g ethanol containing 0.1% w/v bromothymol blue in a sealed 10 ml glass vial. The mixture was stirred at 80° C. for 20 minutes to form a homogeneous yellow coloured oil phase.

### Example 14(N)

2.0176 g MCT oil was mixed with 0.0611 g CMH and 0.1014 g ethanol containing 0.1% w/v bromothymol blue in a sealed 10 ml glass vial. The mixture was stirred at 80° C. for 10 minutes to form a homogeneous yellow coloured oil phase.

### Example 15(O)

2.7904 g soybean oil was mixed with 0.0088 g CMH and 0.1544 g ethanol containing 0.1% w/v bromothymol blue in a sealed 10 ml glass vial. The mixture was stirred at 80° C. for 10 minutes to form a homogeneous yellow coloured oil phase.

### Example 16(P; Reference Solution)

0.028 g ethanol containing 0.1% w/v bromothymol blue was dissolved in 15 ml of the buffer solution and used as a reference solution, against which the release of bromothymol blue from mixtures 1 to 0 was compared.

Release Studies

1 ml of the mixture A to H, respectively, and 1 to 0, respectively, was added to a 25 ml glass beaker containing 15 ml of the buffer solution at a temperature of 37° C. The content was stirred with a magnet throughout the release period and a 1 ml sample was taken for absorbance measurements at 664 nm (A-H) and at 617 nm (I-P) after 0.5, 1, 2, 3, 4, and 20 hours, respectively. Each sample volume was immediately replaced by the same volume of buffer solution.

The results of these release experiments are shown in Table 1 (methylene blue as marker substance) and Table 2 (bromothymol blue as marker substance), respectively.

TABLE 1
Release studies with methylene blue

| Time (h) | | | | | | | Reference |
|---|---|---|---|---|---|---|---|
| Mixture | 0.5 | 1 | 2 | 3 | 4 | 20 | H |
| A | 0.000 | 0.000 | 0.000 | 0.000 | 0.001 | .016 | 0.441 |
| B | 0.020 | 0.013 | 0.014 | 0.020 | 0.025 | 0.038 | |
| C | 0.057 | 0.059 | 0.076 | 0.079 | 0.080 | 0.150 | |
| D | 0.071 | 0.077 | 0.088 | 0.096 | 0.103 | 0.157 | |
| E | 0.000 | 0.000 | 0.000 | 0.000 | 0.000 | 0.000 | |
| F | 0.005 | 0.006 | 0.010 | 0.012 | 0.012 | 0.29 | |
| G | 0.000 | 0.000 | 0.002 | 0.003 | 0.002 | 0.012 | |

A: CMH/soybean oil/ethanol with 0.1% methylene blue 3.0/92.2/4.8% w/w
B: PE/soybean oil/ethanol with 0.1 methylene blue 20.0/75.1/4.9% w/w
C: Soybean oil/EtOH with 0.1% Methylene blue 95.0/5.0% w/w
D: MCT oil/ethanol with 0.1% methylene blue 95.0/5.0% w/w
E: CMH/castor oil/ethanol with 0.1% methylene blue 3.0/92.2/4.8% w/w
F: CMH/MCT oil/ethanol with 0.1% methylene blue 3.0/91.7/5.3% w/w
G: CMH/soybean oil/ethanol with 0.1% methylene blue 0.30/94.88/4.82% w/w

TABLE 2
Release studies with bromothymol blue

| Time (h) | | | | | | | Reference |
|---|---|---|---|---|---|---|---|
| Mixture | 0.5 | 1 | 2 | 3 | 4 | 20 | P |
| I | 0.000 | 0.004 | 0.004 | 0.003 | 0.001 | 0.006 | 0.113 |
| J | 0.002 | 0.003 | 0.001 | 0.000 | 0.002 | 0.002 | |
| K | 0.021 | 0.029 | 0.046 | 0.062 | 0.052 | 0.070 | |
| L | 0.040 | 0.053 | 0.061 | 0.060 | 0.052 | 0.064 | |
| M | 0.004 | 0.008 | 0.011 | 0.012 | 0.012 | 0.025 | |
| N | 0.008 | 0.012 | 0.016 | 0.024 | 0.026 | 0.042 | |
| O | 0.010 | 0.011 | 0.021 | 0.025 | 0.031 | 0.058 | |

I: CMH/soybean oil/ethanol with 0.1% bromothymol blue 3.0/91.5/5.5% w/w
J: PE/soybean oil/ethanol with 0.1% bromothymol blue 19.9/75.1/4.9% w/w
K: soybean oil/ethanol with 0.1% bromothymol blue 95.0/5.0% w/w
L: MCT oil/ethanol with 0.1% bromothymol blue 95.0/5.0% w/w
M: CMH/castor oil/ethanol with 0.1% bromothymol blue 3.0/91.9/5.1% w/w
N: CMH/MCT oil/ethanol with 0.1% bromothymol blue 2.8/92.5/4.7% w/w
O: CMH/soybean oil/ethanol with 0.1% bromothymol blue 0.30/94.47/5.23% w/w

From the tests above it has surprisingly been found that by mixing the triglyceride oil with a polar lipid a strongly improved sustained release of a marker substance can be obtained. C in Table 1 and K in Table 2 contain no polar lipids and the release of the marker substances after 20 hours from these systems was compared to carriers with polar lipids. Table 3 below summarises the results, calculated as percentages of the release from C and K, respectively.

TABLE 3
Release in % of release of C and K after 20 hours

| | A | B | G |
|---|---|---|---|
| C | (C + 3% CMH) | (C + 20% PE) | (C + 0.3% CMH) |
| 100 | 11 | 25 | 8 |
| | I | J | O |
| K | (K + 3% CMH) | (K + 20% PE) | (K + 0.3% CMH) |
| 100 | 9 | 3 | 83 |

Additional Experiments

Additional experiments have been made on the CMH-system to emphasize the potential of the system. To show how one can control the behaviour of the system by altering the triglyceride oil, the amount of polar lipid and also the influence on the system from the incorporated marker-substance an experimenterial design, a factorial design was made. The triglyceride oils were sesame seed oil, MCT oil (medium chain triglyceride oil) and extracted castor oil, the polar lipid was CMH (monohexosylceramide) at three different levels 0.5, 1.6 and 5.0% w/w. The amount of ethanol in each sample was 10% w/w and the rest was the oil. The marker substances were bromothymol blue, which is slightly soluble in water, and safranine O, which is soluble in water. The number of experiments was 18.

The following materials were used:

Sesame seed oil from Croda Oleochemicals, England;

MCT oil (medium chain triglyceride oil) from Croda Oleochemicals, England;

Castor oil, extracted, (triricineolin), RRR, was prepared by Scotia LipidTeknik AB from castor oil from Karlshamns AB, Sweden;

CMH (monohexosylceramide) was prepared from whey concentrate by means of chromatographic fractionation to a purity of >98% by Scotia LipidTeknik AB, Sweden;

Bromothymol blue, BTB, grade “indicator”, was purchased from KEBO Lab AB, Sweden;

Safranine O, SafO, Basic Red 2, [477-73-6] was purchased from Labora Chemicals, Sweden;

Spectra/Por® Membrane MWCO 6000-8000 with weighted closures, KEBO Lab AB, Sweden.

Dissolution Equipment

A conventional USP dissolution bath, PTWS, has been modified so it can be used with lesser volumes. The lids to the original vessels have been modified so that a 50 ml round bottomed flask can be placed in them. The original paddles are made smaller to fit these new vessels which hang inside the original vessels which are filled with water. The temperature in the water bath is set to 38.5° C., which corresponds to a temperature of 37.2-37.3° C. inside the 50 ml vessel.

Preparation of the Formulations

For each formulation the oils were mixed with CMH and ethanol containing 0.3% w/w bromothymol blue, BTB, or 0.1% w/w Safranine O, SafO, in a sealed 10 ml glass vial. The mixtures were stirred at 80° C. for 10 minutes to form a homogeneous yellow coloured (BTB) or ruby-red coloured (SafO) oil phase. The oil phases were transferred to 2 ml syringes before they were brought back to room temperature. The composition of the formulations discussed below under Results from the release studies is shown in Table 4.

TABLE 4
Composition of the formulations

| | CMH | Oil | EtOH | Marker |
|---|---|---|---|---|
| Formulation | % (w/w) | % (w/w) | % (w/w) | substance |
| Q | 1.6 | MCT, 88.4 | 10.0 | BTB |
| R | 1.6 | RRR, 88.4 | 10.0 | BTB |
| S | 1.6 | Sesame, 88.4 | 10.0 | BTB |
| T | 5.0 | Sesame, 85.0 | 10.0 | BTB |
| U | 1.6 | RRR, 88.4 | 10.0 | SafO |
| V | 5.0 | RRR, 85.0 | 10.0 | SafO |
| W | 1.6 | MCT, 88.4 | 10.0 | SafO |
| X | 1.6 | Sesame, 88.4 | 10.0 | SafO |

Release Studies

25 ml dissolution media was administered to the 50 ml inner vessels and allowed to reach the right temperature, approximately 37.3° C., before the experiments start. The stirring rate was 80 rpm. The Spectra/Por® Membrane should be soaked in distilled water for at least 30 minutes before use. Approximately 0.4 g of the lipid mixture was weighed in a piece of the Spectra/Por® Membrane. The membrane was locked at both ends with weighted closures. The formulation in its membrane was put into the medium. Sample was taken after specific times. The dissolution medium was used as a blank on the UV-spectrophotometer. To take a sample the peristaltic pump which is adherent to the flow cuvette system of the UV-spectrophotometer was used. The absorbance was measured at 521 nm (SafO) and 617 nm (BTB). The flow cuvette was filled with sample and the absorbance was measured, afterwards the pump was allowed to work in the reverse direction and the sample was returned to the inner vessel. The cuvette system was then rinsed thoroughly with dissolution media, that is buffer solution.

Results from the Release Studies

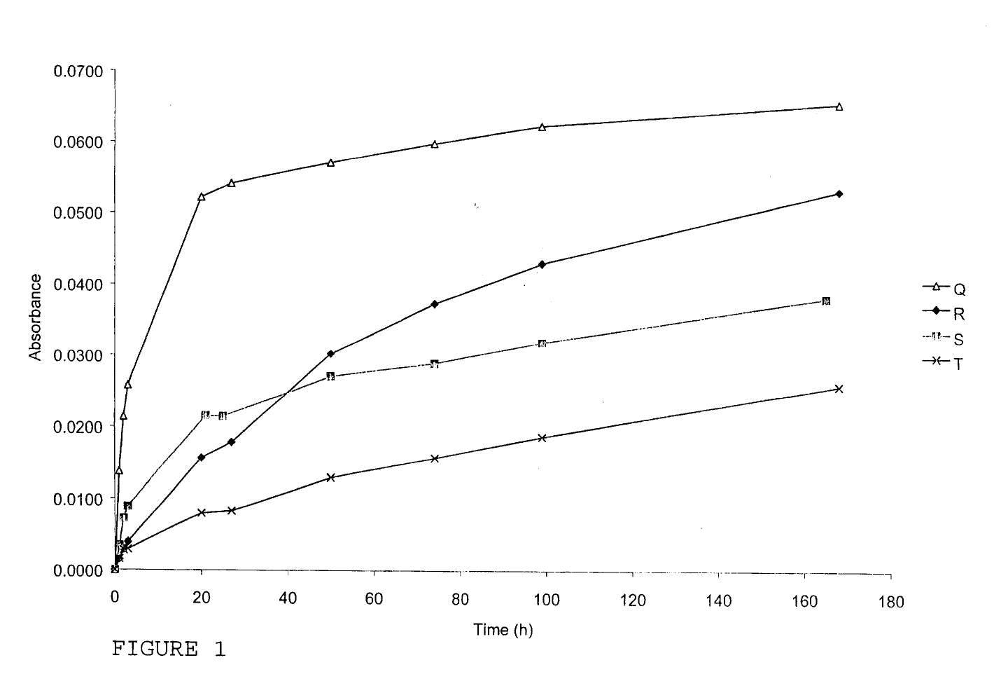

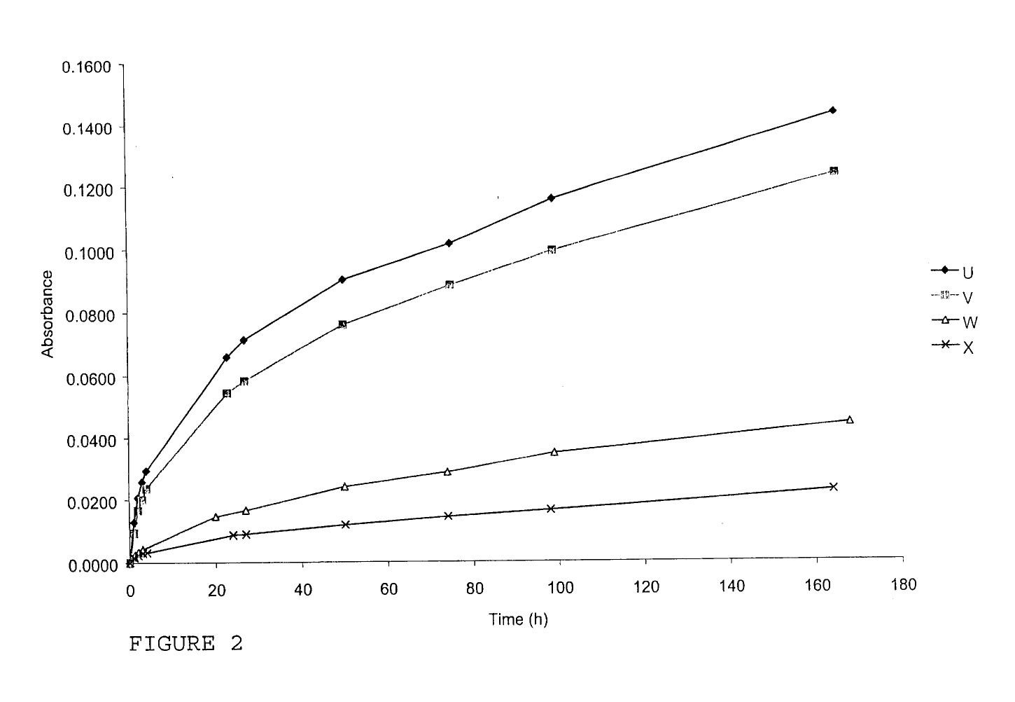

The dissolution profiles from the experiments stated in Table 4 are shown in FIG. 1 and FIG. 2.

The chosen examples show how the dissolution profiles varies depending on the oil, the amount of CMH and also on the marker substance. An evaluation on the dissolution curves from all the experiments with MLR (Multiple Linear Regression) show that the choice of oil, the amount of CMH and the marker substance all are significant for the dissolution profile one will get.

Conclusions from the Experiments

The capacity of the lipid carrier to incorporate drug substances is clearly demonstrated in Experiments 25 to 30, in which about 4-6% by weight of six structurally very different drug substances successfully have been incorporated. In all cases the resulting composition is injectable.

The experiments clearly confirms the surprising observation that when the non-polar lipid is combined with the polar lipid a dramatic effect of improved sustained release of the marker substances from the lipid carrier is observed.

The first experiments also clearly demonstrate that the composition of the polar lipid and the nonpolar lipid in the lipid carrier is the determining factor for the release rate of a specific incorporated substance. From Table 3 it is also obvious that the release rate varies with the composition of the lipid carrier. PE as the polar lipid results in a different release rate than CMH. Different concentrations of CMH give different release rates, which means that the rate can be predicted from the composition. The additional experiments show that the composition of the lipid carrier is the determining factor for the release profile of a specific incorporated substance.

It is also clear from the experiments that the two marker substances are released at different rates from the same lipid carrier, and that these two marker substances are most effectively retained, respectively, by two different lipid carriers. The results from the two studied systems in the additional experiments, BTB and SafO, show that the composition of the system can be modified to suit the incorporated substance and the desired behaviour of the system.

From the experiments, observations and conclusions summarised above it is obvious that the characteristics of the invention make it especially suitable as a pharmaceutical carrier for sustained release of incorporated bioactive compounds. The composition and proportions of the lipids in the carrier can be adjusted to facilitate the incorporation of various bioactive compounds and to control their release rate from the carrier.

1. A lipid carrier composition for controlled release of a bioactive substance, comprising at least one triglyceride oil, and at least one polar lipid selected from the group consisting of phosphatidylethanolamine and monohexosylceramide, and ethanol, characterised in that the carrier composition has the ability to form a cohesive structure which is retained in an aqueous environment.

2. A lipid carrier according to claim 1, characterised in that the acyl groups of the polar lipid, which can be the same or different, are derived from unsaturated or saturated fatty acids or hydroxy fatty acids having 12-28 carbon atoms.

3. A lipid carrier according to claim 1 or 2, characterised in that the phosphatidylethanolamine is egg-PE or dioleyl-PE.

4. A lipid carrier according to claims 1 or 2, characterised in that the monohexosylceramide is obtained from milk.

5. A lipid carrier according to any of claims 1-4, characterised in that the triglyceride oil is selected from the group consisting of soybean oil, sesame oil, medium chain triglyceride oil, castor oil or a mixture thereof.

6. A lipid carrier composition according to any of claims 1-5, characterised in consisting of 60-98% by weight of a triglyceride in combination with 0.1-40% by weight of at least one polar lipid selected from the group consisting of phosphatidylethanolamine and monohexosylceramide, and 0.1-30% by weight of ethanol.

7. A lipid carrier according to claim 6, characterised in that the content of phosphatidylethanolamine is 5-40% by weight of the total carrier composition, preferably 10-25%.

8. A lipid carrier according to claim 6, characterised in that the content of monohexosylceramide is 0.1-25% by weight of the total carrier composition, preferably 0.3-10%.

9. A lipid carrier according to any of claims 1-8, which in addition contains one or more additives selected from the group consisting of glycerol, polyethylene glycols, propylene glycol, fatty alcohols, sterols, monoglycerides, tetraglycol, propylene carbonate and copolymers of polyethylene oxide and polypropylene oxide, and mixtures thereof in an amount of up to 30% by weight of the total carrier composition.

10. Use of a lipid carrier according to any of claims 1-9 for the preparation of a depot formulation for injection for controlled release of a bioactive substance in vivo.

11. Use of a lipid carrier according to any of claims 1-9 for the preparation of an oral formulation for controlled release of a bioactive substance in vivo.

12. Use of a lipid carrier according to any of claims 1-9 for the preparation of an ocular, dental or dermal formulation for controlled release of a bioactive substance in vivo.

13. A pharmaceutical composition for controlled release of a bioactive substance, which composition consists of a) a lipid carrier comprising at least one triglyceride oil in combination with at least one polar lipid selected from the group consisting of phosphatidylethanolamine and monohexosylceramide, and ethanol, which carrier has the ability to form a cohesive structure which is retained in an aqueous environment, and b) a bioactive substance dissolved or dispersed in said carrier.

14. A pharmaceutical composition according to claim 13, characterised in that the lipid carrier consists of 60-98% by weight of a triglyceride in combination with 0.1-40% by weight of at least one of phosphatidylethanolamine and monohexosylceramide, and 0.1-30% by weight of ethanol, based on the total weight of the carrier, in addition to the bioactive substance.

15. A pharmaceutical composition according to claim 13 or 14, which in addition contains one or more additives selected from the group consisting of glycerol, polyethylene glycols, propylene glycol, fatty alcohols, sterols, monoglycerides, tetraglycol, propylene carbonate and copolymers of polyethylene oxide and polypropylene oxide, and mixtures thereof.

16. A pharmaceutical composition according to any of claims 13-15, characterised in that the bioactive substance is selected from the group consisting of neuroleptic, antidepressive, antipsychotic, antibiotic, antimicrobial, antitumour, and anti-Parkinson drugs, hormones, minerals and vitamins.
